(19) United States
(12) Patent Application Publication
(10) Pub. No.: US 2004/0037840 A1
(43) Pub. Date: Feb. 26, 2004

(54) Novel therapeutic vaccine formulations

(76) Inventors: Anne Mette Beier, Horsholm (DK); Anand Gautam, Horsholm (DK); Soren Mouritsen, Horsholm (DK)

Correspondence Address: BIRCH STEWART KOLASCH & BIRCH, PO BOX 747, FALLS CHURCH, VA, 22040-0747, US

(21) Appl. No.: 09/984,092

(22) Filed: Oct. 26, 2001

Related U.S. Application Data
(60) Provisional application No. 60/245,166, filed on Nov. 3, 2000.

(30) Foreign Application Priority Data
Oct. 27, 2000 (DK) PA 2000 01606
Jun. 18, 2001 (DK) PA 2001 00936

(51) Int. Cl. A61K 39/00

(52) U.S. Cl. 424/185.1; 514/55

(57) ABSTRACT

The present invention relates to a novel method and formulation for the induction of immune responses against polypeptide antigens. In particular, the invention provides a method and formulation for induction of cytotoxic T cell responses against a polypeptide antigen of choice. The formulations are characterized by containing chitosan in admixture with the polypeptide antigen, preferably in the form of microparticles that may be cross-linked.

### FIELD OF THE INVENTION

The present invention relates to novel methods for combatting diseases, such as cancers, which are characterized by the presence of gene expression products which are non-immunogenic or poorly immunogenic. In particular, the present invention relates to methods for inducing an immune response conducted by cytotoxic T-lymphocytes (CTLs), whereby cells carrying epitopes from the gene expression products are attacked and killed by the CTLs. The invention specifically relates to formulation in chitosan and other chitin-derivatives of selfproteins and “immunogenized” self-proteins in order to provide for enhanced specific immune responses, especially enhanced CTL responses.

Hence, the invention relates to a series of applications of vaccination technology, e.g. within the field of therapeutic vaccination against cancer, but also within the general field of protein vaccination where CTL responses are desired.

### BACKGROUND OF THE INVENTION

The idea of vaccinating against cancer has been around for more than hundred years and has enjoyed recurrent bursts of activity, particularly since the turn of this century.

However, during the past 10 years the understanding of the fundamental molecular mechanisms of the immune response has improved considerably. Among the most important milestones achieved during this period has been the discovery of the still growing list of cytokines and growth factors, the understanding of the mechanisms of interaction between T and B cells as well as the establishment of the cellular antigen processing pathways including the role and structure of the MHC class I and II molecules in antigen presentation. Important discoveries with regard to cancer immunology—although still not fully understood—were also the elucidation of the mechanisms underlying induction of immunological tolerance in a host. All this research has led to a huge amount of efforts in order to develop new treatments for human cancer.

Depending on how tumour immunity is acquired by the patient, immunotherapy regimens can be categorised as either passive or active. In passive immunotherapy regimens the patient passively receives immune components such as cytokines, antibodies, cytotoxic T-cells, or lymphocyte activated killer (LAK) cells. In contrast, active specific immunotherapy protocols encompass actively inducing tumour immunity by vaccination with the tumour cell or its antigenic components. This latter form of treatment is preferred because the immunity is prolonged.

Passive and active cancer vaccines have focussed on inducing either humoral or cellular immune responses. For active vaccines it is well established that induction of CD4 positive T helper cells is necessary in order to secondarily induce either antibodies or cytotoxic CD8 positive T cells.

Passive Vaccination with Antibodies

Since the discovery of the monoclonal antibody technology in the mid-seventies, a large number of therapeutic monoclonal antibodies directed against tumour specific or tumour associated antigens has been developed. Monoclonal antibody therapy, however, gives rise to several serious problems:

Injection of these foreign substances induces an immune response in the patient towards the injected antibodies, which may lead to less efficient treatment as well as to serious allergic side-effects in the patients.

Monoclonal antibodies usually must be administered in large amounts. This is a problem, since the production costs of monoclonal antibodies are huge.

Monoclonal antibodies must be administered via the parenteral route and due to the relatively large amounts needed, the patients frequently must be hospitalised during the treatment.

Injections of monoclonal antibodies must be repeated at rather short intervals (weeks) in order to maintain therapeutic effect.

Monoclonal antibodies are usually not able to activate secondary effector systems of the immune system such as complement, NK-cells or macrophage killing of tumour cells.

The latter disadvantage is of particular importance in cancer therapy and may be an important reason why monoclonal antibody therapy of cancer in several cases has not been particularly successful. The so-called humanised monoclonal antibodies now used by many companies are less immunogenic, but unfortunately they are even less capable of activating the secondary immune effector systems. Furthermore, examples of secondary outgrowth of tumours lacking the original tumour antigen have been observed, since these antibodies do not induce “innocent bystander” effects on tumour cells not carrying the tumour antigen.

The poor effector capability of the monoclonal antibodies has led to the development of monoclonal antibodies chemically conjugated to different toxins and radioisotopes. Pharmacia Upjohn AB has e.g. developed a conjugate between a monoclonal tumour specific antibody and the Staphylococcus aureus toxin A with the purpose of activating T cells in the tumour. Medarex Inc. has developed bispecific monoclonal antibodies containing a tumour specific Fab fragment as well as an Fc-receptor specific antibody fragment with the purpose of activating macrophage killing of tumour cells. Both constructs are more effective than the monoclonal antibody alone, but they are also more expensive and immunogenic. Antibodies conjugated to radioisotopes are also expensive as well as immunogenic and other general toxic side-effects are observed.

The appearance of the monoclonal antibody technology was a major step forward which enabled the production of well-defined, high-affinity binding molecules. However, being monoclonal these antibodies only react with a single type of epitope on a tumour antigen. This is the major reason why they usually are not able to activate the complement system or binding to the Fc-receptors of NK-cells and macrophages. These very powerful effector systems usually require the co-localisation of multiple Fc antibody fragments protruding from the antigen.

Other researchers have therefore attempted to use two monoclonal antibodies in combination and this has led to an improved effect. It therefore seems very reasonable instead to attack tumour cells with highly specific polyclonal antibodies directed against a tumour specific, or against (over-expressed) tumour associated antigens or growth factor receptors. Such antibodies would be fully capable of activating the secondary effector systems mentioned above. Furthermore, it is likely that the local inflammatory reaction induced by these effector systems could lead to secondary effects on “innocent bystander” cells not expressing the tumour antigen in question as well as to activation of tumour specific TIL's (tumour infiltrating lymphocytes) in the tumour tissue. Such effects have been observed by Medarex Inc. using their bi-specific monoclonal antibody conjugates.

Since the discovery of the monoclonal antibody technology the potential use of polyclonal antibodies for cancer therapy has not been explored very much (except for the antigens described below). One major reason is that well-defined tumour specific or tumour associated surface antigens only have been characterised within the recent years, but—more importantly—many of these have turned out to be self-antigens and therefore non-immunogenic. Accordingly, xenogenic polyclonal antibodies would necessarily have been used to study the effects. However, such antibodies induce a vigorous immune response towards the injected foreign polyclonal antibodies which rapidly eliminate the therapeutic effects.

Active Vaccination to Induce Antibodies

Recent attempts to induce therapeutic polyclonal autoantibodies in cancer patients by active vaccination have been successful. Vaccines against membrane bound carbohydrate self-antigens (such as the O-linked aberrantly expressed Tn and stn-antigens and the ganglioside liposaccharides GM2 and GD3) have been developed. These small carbohydrate structures are, however, very poor antigens so conjugates of these molecules with carrier molecules such as keyhole limpet haemocyanin (KLH) or sheep mucins (containing Tn- and sTn) must be used. In melanoma patients the induction of anti-GM2 antibodies were associated with a prolonged disease-free interval and overall survival after a minimum follow-up of fifty-one months. Also randomised phase II studies have been conducted on breast cancer patients using a conjugate of sTn and KLH in the DETOX-B adjuvant (BIOMIRA Inc.) showing that sTn immune patients had a significantly longer median survival compared to controls. Another example of the active induction of polyclonal antibodies in cancer is the use of idiotype specific vaccination against B-cell lymphomas, which—although it has been promising—is limited to this cancer type only.

Finally, the US company Aphton Inc. has developed active conjugate vaccines against gonadotropin releasing hormone (GnRH) and gastrin. It has been demonstrated, that this vaccine is capable of controlling the biological activity of these hormones, which also can function as autocrine growth factors for certain tumour cells. Successful phase II clinical trials have been conducted on gastrointestinal cancer patients and phase III clinical trials are underway.

Cytotoxic T-Cells

It has been clearly demonstrated by several groups that tumour specific cytotoxic T cells (CTL's) are present in many tumours. These CTL's are termed tumour infiltrating lymphocytes (TIL's). However, these cells are somehow rendered non-responsive or anergic by several different possible mechanisms including secretion of immunosuppressive cytokines by the tumour cells, lack of co-stimulatory signals, down regulation of MHC class I molecules etc.

There has been many attempts to isolate the tumour specific HLA class I bound peptides recognised by TILs, and in some cases it has also been successful (e.g. peptides from the melanoma associated antigens). Such peptides have been used to induce a tumour specific immune response in the host, but the practical use of tumour specific peptides in vaccines is restricted to a limited segment of the population due to the narrow HLA class I binding specificity of the peptides. Furthermore, it is usually relatively difficult to evoke a CTL response in vivo using synthetic peptides due to the low biological half-life of these substances as well as the difficulties with exogenous priming of MHC class I molecules.

Many other approaches have been attempted in order to evoke a tumour specific CTL response including the use of cytokines (e.g. IL-2, IFN-γ, IL-6, IL-4, IL-10 or GM-CSF) or co-stimulatory molecules (B7) either in soluble form or expressed by the transfected tumour cell. Furthermore, immunisations with allogenic or autologous whole cells, or of tumour antigens prepared in specialised adjuvants designed to present the antigen via the MHC class I antigen presentation route, or tumour antigens expressed in e.g. vaccinia vectors etc. have been used with varying success. Still the general belief among tumour immunologists is therefore that one of the best ways to eliminate tumours would be to induce a strong specific anti-tumour CTL response.

Apart from the fact that these treatments usually are very expensive and difficult to reproduce, it has also turned out to be difficult to obtain a good immune response towards the tumour since many of the tumour associated antigens are true self-proteins to which most T cells appear to be tolerant. Therefore, it seems necessary to induce a controlled cellular autoimmune condition in the patient.

### OBJECT OF THE INVENTION

It is an object of the present invention to provide improved methods and agents for inducing immune responses in host organisms against undesirable antigens, e.g. tumour antigens. In particular it is an object of the present invention to provide formulations of proteinaceous antigens that are capable of inducing effective CTL responses against these antigens.

### SUMMARY OF THE INVENTION

Presentation of antigens has dogmatically been thought of as two discrete pathways, a class II exogenous and a class I endogenous pathway.

Briefly, a foreign protein from outside the cell or from the cell membrane is taken up by the APC as an endosome which fuses with an intracellular compartment which contains proteolytic enzymes and MHC class II molecules. Some of the produced peptides bind to class II, which then are translocated to the cell membrane.

The class I endogenous pathway is characterised by the predominant presentation of cytosolic proteins. This is believed to occur by proteasome mediated cleavage followed by transportation of the peptides into the endoplasmic reticulum (ER) via TAP molecules located in the membrane of the ER. In ER the peptides bind to class I followed by transportation to the plasma membrane.

However, these 2 pathways are not fully distinct. For example it is known that dendritic cells and to some extend macrophages are capable of endocytosing (pinocytosing) extracellular proteins and subsequently present them in the context of MHC class I. It has also previously been demonstrated that using specialised administration routes, e.g. by coupling to iron oxide beads, exogenous antigens are capable of entering the Class I pathway (Rock, 1996). This mechanism seems central, because of the importance of a concomitant expression of both class I and class II on the same APC to elicit a three cell type cluster. This three cell type cluster of interaction has been proposed by Mitchison (1987) and later by other authors. They showed the importance of concomitant presentation of class I and class II epitopes on the same APC. According to the recently described mechanism for CTL activation (cf. Lanzavecchia, 1998, Nature 393: 413, Matzinger, 1999, Nature Med. 5: 616, Ridge et al., 1998, Nature 393: 474, Bennett et al., 1998, Nature 393: 478; Schoenberger et al., 1998, Nature 393: 480, Ossendrop et al., 1998, J. Exp. Med 187: 693, and Mackey et al., 1998, J. Immunol 161: 2094), professional APCs presenting antigen on MHC class II are recognized by T helper cells. This results in an activation of the APC (mediated by interaction by CD40L on the T helper cell and CD40 on the APC). This enables the APC to directly stimulate CTLs which are thereby activated. Cf. also FIG. 2.

It has previously been demonstrated that insertion of a foreign MHC class II restricted T helper cell epitope into a self-antigen results in the provision of an antigen capable of inducing strong cross-reactive antibody responses directed against the non-modified self-antigen (cf. applicant's WO 95/05849). It was shown that the autoantibody induction is caused by specific T cell help induced by the inserted foreign epitope.

However, we have come to the conclusion that modified self-antigens—with the aid of appropriate adjuvants—ought to be capable of also inducing strong CTL responses against MHC class I restricted self-epitopes and hence the technology described in WO 95/05849 can be adapted to also provide vaccination against intracellular and other cell-associated antigens which have epitopes presented in the context of MHC Class I. This inventive concept is the subject matter of WO 00/20027.

The autovaccine technology described in WO 95/05849 has the effect that specific T cell help is provided to self-reactive B cells when a modified self-antigen is administered for uptake into the MHC class II antigen processing pathway (cf. FIG. 1, and Dalum I et al., 1996, J. Immunol. 157: 4796-4804 as well as Dalum I et al., 1999, Nature Biotechnol. 17: 666-669). It was shown that potentially self-reactive B-lymphocytes recognizing self-proteins are physiologically present in normal individuals. However, in order for these B-lymphocytes to be induced to actually produce antibodies reactive with the relevant self-proteins, assistance is needed from cytokine producing T-helper lymphocytes (T_{H}-cells or T_{H}-lymphocytes) Normally this help is not provided because T-lymphocytes in general do not recognize T-cell epitopes derived from selfproteins when presented by antigen presenting cells (APCs). However, by providing an element of “foreignness” in a self-protein (i.e. by introducing an immunologically significant modification), T-cells recognizing the foreign element are activated upon recognizing the foreign epitope on an APC (such as, initially, a mononuclear cell). Polyclonal B-lymphocytes (which present T-cell epitopes) capable of recognising self-epitopes on the modified self-protein also internalise the antigen and subsequently presents the foreign T-cell epitope(s) thereof, and the activated T-lymphocytes subsequently provide cytokine help to these self-reactive polyclonal B-lymphocytes. Since the antibodies produced by these polyclonal B-lymphocytes are reactive with different epitopes on the modified polypeptide, including those which are also present in the native polypeptide, an antibody cross-reactive with the non-modified self-protein is induced. In conclusion, the T-lymphocytes can be led to act as if the population of polyclonal B-lymphocytes have recognised an entirely foreign antigen, whereas in fact only the inserted epitope(s) is/are foreign to the host. In this way, antibodies capable of cross-reacting with non-modified self-antigens are induced.

As mentioned above, CTL's also require specific T cell help, although the mechanism for this is still not completely clear. However, the invention disclosed in WO 00/20027 has demonstrated that self-proteins containing foreign MHC class II epitopes and entering the MHC class I antigen processing pathway of e.g. macrophages and dendritic cells induces a strong CTL response against subdominant epitopes in the self-protein. In essence there are two ways that such self-protein analogues can enter the Class I pathway: By use of nucleic acid immunization and by use of a formulation/adjuvant which effects uptake of the analogues into APCs so as to enter the Class I pathway.

The present invention primarily provides for such formulations/adjuvants that utilise various forms of chitin derivatives such as chitosan.

In conclusion, a vaccine constructed using the technology outlined above will induce a humoral autoantibody response with secondary activation of complement and antibody dependent cellular cytotoxicity (ADCC) activity. It is also expected that it will induce a cytotoxic T cell response directed against e.g. a tumour specific membrane antigen, but it is according to the present invention concluded that this CTL response will be greatly facilitated if the polypeptide construct in question is formulated with a chitin derivative as described below.

Hence, in the broadest and most general scope, the present invention relates to a method for inducing or enhancing an immune response against a polypeptide antigen in an animal, including a human being, said polypeptide antigen being weakly immunogenic or non-immunogenic in the animal, the method comprising administering, to the animal, the polypeptide antigen or at least one variant thereof which includes foreign T-helper Cell epitopes (TH epitopes) or at least one nucleic acid fragment encoding the polypeptide antigen or the at least one variant, wherein the polypeptide antigen or variant thereof or the nucleic acid fragment is formulated with chitosan.

In a more specific variant, this method comprises effecting simultaneous presentation by antigen presenting cells (APCs) from the animal's immune system of an immunogenically effective amount of

1) at least one CTL epitope derived from the polypeptide antigen and/or at least one B-cell epitope derived from the polypeptide antigen, and

2) at least one first T helper cell epitope (TH epitope) which is foreign to the animal.

In a still further specific variant of the inventive method, the polypeptide antigen is a cell-associated polypeptide antigen which is sought down-regulated by inducing a specific cytotoxic T-lymphocyte (CTL) response against cells carrying the polypeptide antigen on their surface or harbouring the polypeptide antigen in their intracellular compartment, the method comprising effecting, in the animal, simultaneous presentation by a suitable antigen presenting cell (APC) of

1) at least one CTL epitope derived from the polypeptide antigen, and

2) at least one first T-helper lymphocyte (TH) epitope which is foreign to the animal.

The invention also relates to compositions comprising chitosan and a number of specific antigens or immunogenic variants of these antigens.

### LEGENDS TO THE FIGURE

FIG. 1: The traditional AutoVac concept. A: Tolerodominant self-epitopes presented on MHC class II on an antigen presenting cell (APC) are ignored due to depletion in the T helper cell (Th) repertoire (T helper cell indicated with dotted lines). Inserted foreign immunodominant T cell epitopes presented on MHC class II activate T helper cells and B cells (B) specific for native parts of the self-protein presenting foreign immunodominant T cell epitopes on MHC class II are activated by the cytokine help provided by the T helper cell.

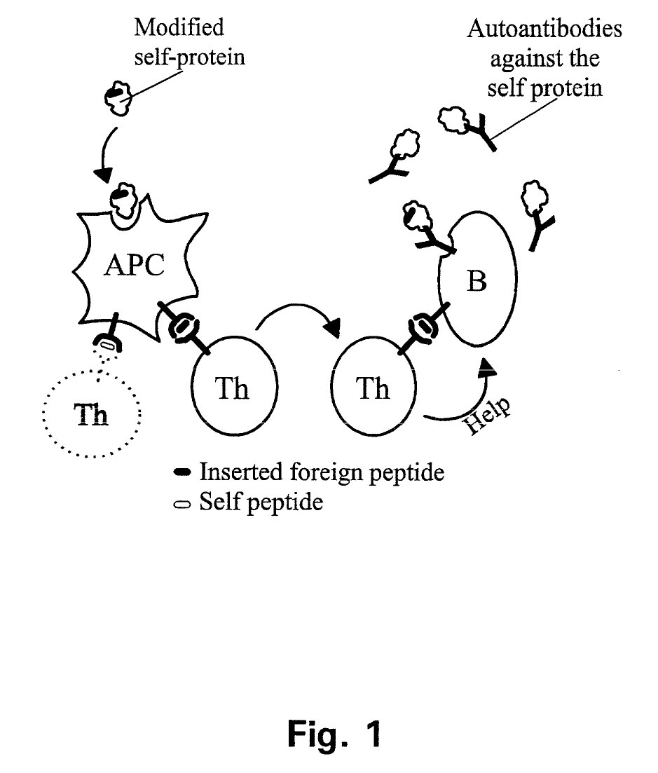

FIG. 2: The AutoVac concept for inducing a CTL response. Inserted foreign immunodominant T cell epitopes presented on MHC class II activate T helper cells. CTL's recognising subdominant self-epitopes presented on MHC class I are activated by the adjacent activated T helper cell.

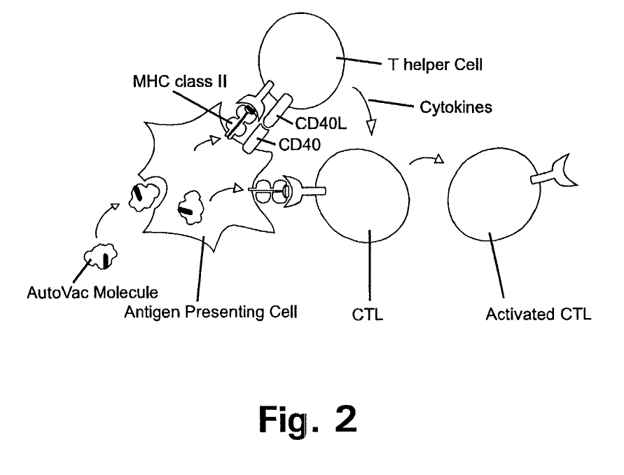

### DETAILED DISCLOSURE OF THE INVENTION

Definitions

In the following a number of terms used in the present specification and claims will be defined and explained in detail in order to clarify the metes and bounds of the invention.

“Chitosan”, B (1,4) 2-amino-2-deoxy-D-glucose, denotes chitin and chitin-derived polysaccharides comprising co-polymers of glucosamine and N-acetylglucosamine. Different chitosans are characterized by molecular weight, viscosity and degree of deacetylation compared to chitin (chitosan is obtained by alkaline hydrolysis of aminoacetyl groups of chitin). The term chitosan also includes within its scope “chitosan based compounds”, i.e. derivatives or analogues of chitin and chitosan that are capable of forming suitable compositions with a polypeptide or a nucleic acid. Such an analogue of derivative may be a modified chitosan or chitin, where the modification serves to alter the physical, chemical or physiological properties thereof. Such an analogue can be formed by non-covalent adherence due to electrostatic and/or hydophilic and/or hydrophobic interactions or by covalent bonding to chitosan or chitin. Examples of analogues include, but are not limited to, chitosan or chitin modified by having bound thereto specific or non-specific targeting ligands and/or membrane permeabilisation agents and/or endosomolytic agents and/or nuclear localisation signals. Other examples are derivatized chitin or chitosan or the above mentioned analogues, i.e. O-acetylated and/or N-acetylated and/or N-trimmethylated chitin, chitosan, or analogues. Finally, also included are salts of all these chitosan-based compounds, e.g. sulfate, phosphate, glutamate, chloride and tripolyphosphate salts (Berthold et al. 1996; Roy et al. 1.999; Mao et al. 2001; van der Lubben et al. 2001).

The chitosan based compounds may advantageously be crosslinked, either naturally or by means of cross-linking or gelling agents such as glutaraldehyde (Akbuga and Durmaz 1994; Aiedeh et al 1997; Jameela et al 1995), formaldehyde or alginate gelation (Liu et al 1997; Alexakis et al 1995; Polk et al 1994).

A “cell-associated polypeptide antigen” is in the present specification and claims intended to denote a polypeptide which is confined to a cell which is somehow related to a pathological process. Furthermore, the cell presents CTL epitopes of the polypeptide antigen bound to MHC Class I molecules on its surface. Cell-associated polypeptide antigens can therefore be truly intracellular antigens (and thereby unreachable for a humoral immune response) or antigens bound to the surface of the cells. The cell-associated polypeptide antigen can be the product of the cell's own gene expression, of an intracellular parasite, of a virus, or of another cell. In the latter case the polypeptide antigen is subsequently associated with the cell which is involved in the pathological process. Many of the polypeptide antigens against which the present invention is aimed, are cell-associated antigens. The term also includes within its scope a fragment of a protein, that is, the term “polypeptide antigen” denotes a continuous amino acid sequence found in a native protein.

The terms “T-lymphocyte” and “T-cell” will be used interchangeably for lymphocytes of thymic origin which are responsible for various cell mediated immune responses as well as for effector functions such as helper activity in the humoral immune response. Likewise, the terms “B-lymphocyte” and “B-cell” will be used interchangeably for antibody-producing lymphocytes.

An “antigen presenting cell” (APC) is a cell which presents epitopes to T-cells. Typical antigen-presenting cells are macrophages, dendritic cells and other phagocytizing and pinocytizing cells. It should be noted that B-cells also functions as APCs by presenting T_{H} epitopes bound to MCH class II molecules to T_{H} cells but when generally using the term APC in the present specification and claims it is intended to refer to the above-mentioned phagocytizing and pinocytizing cells.

“Helper T-lymphocytes” or “T_{H} cells” denotes CD4 positive T-cells which provide help to B-cells and cytotoxic T-cells via the recognition of T_{H} epitopes bound to MHC Class II molecules on antigen presenting cells.

The term “cytotoxic T-lymphocyte” (CTL) will be used for CD8 positive T-cells which require the assistance of T_{H} cells in order to become activated.

A “specific” immune response is in the present context intended to denote a polyclonal immune response directed predominantly against a molecule or a group of quasi-identical molecules or, alternatively, against cells which present CTL epitopes of the molecule or the group of quasi-identical molecules.

A “weak or non-immunogenic polypeptide antigen” is herein intended to denote polypeptides having the amino acid sequence of the weak protein antigens derived from the animal in question (e.g. a human), but also polypeptides having the amino acid sequence identical to analogues of such proteins isolated from other species are embraced by the term. Also forms of the polypeptides having differing glycosylation patterns because of their production in heterologous systems (e.g. yeasts or other non-mammalian eukaryotic expression systems or even prokaryotic systems) are included within the boundaries of the term. It should, however, be noted that when using the term, it is intended that the polypeptide in question is normally non-immunogenic or only weakly immunogenic in its natural localisation in the animal to be treated.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same weak polypeptide antigen allowing for immunization of the animals with the same immunogen(s). If, for instance, genetic variants of polypeptides exist in different human populations it may be necessary to use different immunogens in these different populations in order to be able to break the autotolerance towards the weak polypeptide antigen in each population.

By the term “down-regulation a polypeptide antigen” is herein meant reduction in the living organism of the amount and/or activity of the antigen in question. The down-regulation can be obtained by means of several mechanisms: Of these, simple interference with the active site in the antigen by antibody binding is the most simple. However, it is also within the scope of the present invention that the antibody binding results in removal of the polypeptide by scavenger cells (such as macrophages and other phagocytizing cells), and even more important, that cells carrying or harbouring the antigen are killed by CTLs in the animal.

The expression “effecting simultaneous presentation by a suitable APC” is intended to denote that the animal's immune system is subjected to an immunogenic challenge in a controlled manner which results in the simultaneous presentation by APCs of the epitopes in question. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with APCs displaying the relevant epitopes in an immunologically effective manner.

The term “immunogenically effective amount” has its usual meaning in the art, i.e. an amount of an immunogen which is capable of inducing an immune response which significantly engages pathogenic agents which share immunological features with the immunogen.

When using the expression that the weak polypeptide antigens have been subjected to a “modification” is herein meant a chemical modification of the polypeptide which constitutes the backbone of the polypeptide in question. Such a modification can e.g. be derivatization (e.g. alkylation) of certain amino acid residues in the amino acid sequence, but as will be appreciated from the disclosure below, the preferred modifications comprise changes of the primary structure of the amino acid sequence.

When discussing “tolerance” and “autotolerance” is understood that since the polypeptides which are the targets of the present inventive method are self-proteins in the population to be vaccinated or proteins which do not result in induction of an effective immune response, normal individuals in the population do not mount an immune response against the polypeptide. It cannot be excluded, though, that occasional individuals in an animal population might be able to produce antibodies against the native polypeptide antigen, e.g. as part of a autoimmune disorder. At any rate, an animal will normally only be autotolerant towards its own polypeptide antigen, but it cannot be excluded that analogues derived from other animal species or from a population having a different phenotype would also be tolerated by said animal.

A “foreign T-cell epitope” is a peptide which is able to bind to an MHC molecule and stimulates T-cells in an animal species. Preferred foreign epitopes are “promiscuous” epitopes (also known as universal epitopes), i.e. epitopes which binds to a substantial fraction of MHC class II molecules in an animal species or population. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same analogue or 2) prepare several analogues wherein each analogue has a different promiscuous epitope inserted. It should be noted that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class Class II molecule and can be presented on the surface of an antigen presenting cell (APC) bound to the MHC Class II molecule.

It follows logically from the above, that non-self proteins often by nature contain foreign T_{H} epitopes, and that introduction of other foreign epitopes may be unnecessary simply because polypeptide fragments of the non-self proteins already include both the necessary CTL epitopes and the necessary foreign T_{H} epitopes.

A “CTL” epitope is a peptide which is able to bind to an MHC class I molecule.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. For instance it is possible to use certain cytokines as a modifying moiety in the analogue (cf. the detailed discussion below), and in such a case, the issue of stability may be irrelevant since the coupling to the analogue provides the stability necessary.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen

Preferred embodiments

In order to induce a CTL response against a cell which presents epitopes derived from the polypeptide antigen on its surface, it is normally necessary that at least one CTL epitope, when presented, is associated with an MHC Class I molecule on the surface of the APC. Furthermore it is preferred that the at least one first foreign T_{H} epitope, when presented, is associated with an MHC Class II molecule on the surface of the APC. Preferred APCs presenting the epitopes are dendritic cells and macrophages, but any pino- or phagocytizing APC which is capable of simultaneously presenting 1) CTL epitopes bound to MHC class I molecules and 2) T_{H} epitopes bound to MHC class II molecules, is a preferred APC according to the invention.

According to the invention, the polypeptide antigen is preferably selected from a tumour-associated antigens and other self-proteins which are related to pathological processes but also viral antigens and antigens derived from an intracellular parasite or bacterium will. It is well-known in the art that such pathogen-associated antigens are often relatively poor immunogens (e.g. antigens from mycobacteria such as Mycobacterium tuberculosis and Mycobacterium leprae, but also from protozoans such as Plasmodium spp.). It is believed that the method of the invention, apart from rendering possible the production of antibody and CTL responses against true self-protein antigens, is capable of enhancing the often insufficient immune response mounted by the organism against such intracellular antigens. Hence, the method of the invention is not limited to induction of immune responses against self-proteins but also to induction of CTL responses against any antigen where this is desired. In this context it is important to note that the chitosan formulations described herein are also useful when combined with native polypeptide sequences (complete or truncated versions of those found in native proteins) as long as the native polypeptide sequence includes the T_{H} epitopes necessary to induce an immune response against the native polypeptide.

Normally, it will be advantageous to confront the immune system with a large fraction of the amino acid sequence of the polypeptide antigen which is the vaccine target, i.a. by using the polypeptide in its natural form. Hence, in a preferred embodiment, presentation by the APC of the CTL epitope and the first foreign T_{H} epitope is effected by presenting the animal's immune system with at least one first analogue of the polypeptide antigen, said first analogue comprising a variation of the amino acid sequence of the polypeptide antigen, said variation containing at least the CTL epitope and the first foreign T_{H} epitope. This is in contrast to e.g. a DNA vaccination strategy where the CTL and T_{H} epitopes are expressed by the same cell but as parts of separate polypeptides; such a DNA vaccination strategy is also an embodiment of the invention, but it is believed that having the two epitopes as part of the same polypeptide will normally enhance the immune response and, at any rate, the provision of only one expression product will be necessary.

In order to maximize the chances of mounting an effective immune response, it is preferred that the above-mentioned first analogue contains a substantial fraction of known and predicted CTL epitopes of the polypeptide antigen, i.e. a fraction of the known and predicted CTL epitopes which binds a sufficient fractions of MHC Class I molecules in a population. For instance, it is preferred that the substantial fraction of known and predicted CTL epitopes in the amino acid sequence of the analogue are recognized by at least 50% of the MHC-I haplotypes recognizing all known and predicted CTL epitopes in the polypeptide antigen, but higher percentages are preferred, such as at least 60, at least 70, at least 80, and at least 90%. Especially preferred is the use of analogues which preserve substantially all known CTL epitopes of the polypeptide antigen are present in the analogue, i.e. close to 100% of the known CTL epitopes. Accordingly, it is also especially preferred that substantially all predicted CTL epitopes of the polypeptide antigen are present in the at least first analogue.

Methods for predicting the presence of CTL epitopes are well-known in the art, cf. e.g. Rothbard et al. EMBO J. 7:93-100 (1988).

As will be apparent from the present specification and claims it is expected that the inventive method described herein will render possible an improved induction of CTL responses against polypeptide antigens.

In cases where the polypeptide antigen is truly intracellular, the induction of a CTL response against cells harbouring the antigen is the only way to achieve its down-regulation by specific immunological means. However, in the case of membrane-associated antigens, it is advantageous to induce an antibody response against the weak polypeptide antigen. However, when raising a humoral immune response against a weak antigen it is preferred to substantially restrict the antibody response to interaction with the parts of the antigen which are normally exposed to possible interaction with antibodies. Otherwise the result would most likely be the induction of an antibody response against parts of the antigen which is not normally engaging the humoral immune system, and this will in turn increase the risk of inducing cross-reactivity with antigens not related to any pathology. One elegant way of obtaining this restriction is to perform nucleic acid vaccination with an analogue of the weak antigen, where the extracellular part thereof is either unaltered or includes a T_{H} epitope which does not substantially alter the 3D structure of the extracellular part of the antigen. As one possible alternative, immunization can be performed with both a CTL directed immunogen and a B-cell directed immunogen where the B-cell directed immunogen is substantially incapable of effecting immunization against the intracellular part of the target antigen (the B-cell directed immunogen could e.g. lack any non-extracellular material from the antigen.

Induction of antibody responses can be achieved in a number of ways known to the person skilled in the art. For instance, the at least one first analogue may comprise a part consisting of a modification of the structure of the polypeptide antigen, said modification having as a result that immunization of the animal with the first analogue induces production of antibodies in the animal against the polypeptide antigen this variant is as mentioned above especially suited for nucleic acid vaccination. Alternatively, the method of the invention can involve effecting presentation to the animal's immune system of an immunogenically effective amount of at least one second analogue of the polypeptide antigen which contains such a modification. A convenient way to achieve that the modification has the desired antibody-inducing effect is to include at least one second foreign T_{H} epitope in the second analogue, i.e. a strategy like the one used for the first analogue.

In the cases where it is desired to also mount an effective humoral immune response, it is advantageous that the first and/or second analogue(s) comprise(s) a substantial fraction of the polypeptide antigen's B-cell epitopes, especially a substantial fraction of such B-cell epitopes which are extracellular in the naturally occurring form of the antigen in the pertinent animal.

The above-discussed variations and modifications of the weak polypeptide antigen can take different forms. It is preferred that the variation and/or modification involves amino acid substitution and/or deletion and/or insertion and/or addition. These fundamental operations relating to the manipulation of an amino acid sequence are intended to cover both single-amino acid changes as well as operations involving stretches of amino acids (i.a. shuffling of amino acid stretches within the polypeptide antigen; this is especially interesting when the antigen is a true intracellular antigen, since only considerations concerning preservation of CTL epitopes are relevant). It will be understood, that the introduction of e.g. one single amino acid insertion or deletion may give rise to the emergence of a foreign T_{H} epitope in the sequence of the analogue, i.e. the emergence of an MHC Class-I molecule binding sequence. However, in most situations it is preferable (and even necessary) to introduce a known foreign T_{H} epitope, and such an operation will require amino acid substitution and/or insertion (or sometimes addition in the form of either conjugation to a carrier protein or provision of a fusion polypeptide by means of molecular biology methods. It is preferred that the number of amino acid insertions, deletions, substitutions or additions is at least 2, such as 3, 4, 5, 6, 7, 8, 9, 10, 11, 12, 13, 14, 15, 16, 17, 18, 19, 20, and 25 insertions, substitutions, additions or deletions. It is furthermore preferred that the number of amino acid substitutions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 30.

Preferred embodiments of the invention includes modification by introducing at least one foreign immunodominant T_{H} epitope. It will be understood that the question of immune dominance of a T-cell epitope depends on the animal species in question. As used herein, the term “immunodominance” simply refers to epitopes which in the vaccinated individual/population gives rise to a significant immune response, but it is a well-known fact that a T-cell epitope which is immunodominant in one individual is not necessarily immunodominant in another individual of the same species, even though it may be capable of binding MHC-II molecules in the latter individual. True immune dominant T_{H} epitopes are those which, independent of the polypeptide wherein they form a subsequence; give rise to activation of T_{H} cells—in other words, some T_{H} epitopes have, as an intrinsic feature; the characteristic of substantially never being cryptic since they are substantially always processed by APCs and presented in the context of an MHC II molecule on the surface of the APC.

Another important point is the issue of MHC restriction of T-cell epitopes. In general, naturally occurring T-cell epitopes are MHC restricted, i.e. a certain peptides constituting a T-cell epitope will only bind effectively to a subset of MHC Class II molecules. This in turn has the effect that in most cases the use of one specific T-cell epitope will result in a vaccine component which is only effective in a fraction of the population, and depending on the size of that fraction, it can be necessary to include more T-cell epitopes in the same molecule, or alternatively prepare a multi-component vaccine wherein the components are variants of the antigen which are distinguished from each other by the nature of the T-cell epitope introduced.

If the MHC restriction of the T-cells used is completely unknown (for instance in a situation where the vaccinated animal has a poorly defined MHC composition), the fraction of the population covered by a specific vaccine composition can be determined by means of the following formula

$${f}_{population} = 1 - {\prod }_{i = 1}^{n} ( 1 - {p}_{i} ) \qquad ( II )$$

where Pi is the frequency in the population of responders to the i^{th} foreign T-cell epitope present in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give

1−0.2×0.3×0.4=0.976

i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = 1 - {\prod }_{j = 1}^{3} {( 1 - {ϕ}_{j} )}^{2} \qquad ( III )$$

wherein φⱼ is the sum of frequencies in the population of allelic haplotypes encoding MHC molecules which bind any one of the T-cell epitopes in the vaccine and which belong to the j^{th} of the 3 known HLA loci (DP, DR and DQ); in practice, it is first determined which MHC molecules will recognize each T-cell epitope in the vaccine and thereafter these are listed by type (DP, DR and DQ)—then, the individual frequencies of the different listed allelic haplotypes are summed for each type, thereby yielding φ₁, φ₂, and φ₃.

It may occur that the value pᵢ in formula II exceeds the corresponding theoretical value pᵢ:

$${π}_{i} = 1 - {\prod }_{j = 1}^{3} {( 1 - {v}_{j} )}^{2} \qquad ( IV )$$

wherein vⱼ is the sum of frequencies in the population of allelic haplotype encoding MHC molecules which bind the i^{th} T-cell epitope in the vaccine and which belong to the j^{th} of the 3 known HLA loci (DP, DR and DQ). This means that in 1-πᵢ of the population is a frequency of responders of f_{residual}_{_{—}}ᵢ=(Pᵢπᵢ−πᵢ/(1−pᵢ). Therefore, formula III can be adjusted so as to yield formula V:

$${f}_{population} = 1 - {\prod }_{j = 1}^{3} {( 1 - {ϕ}_{j} )}^{2} + ( 1 - {\prod }_{i = 1}^{n} ( 1 - {f}_{residual\_i} ) ) \qquad ( V )$$

where the term 1−f_{residual}_{_{—}}ᵢ is set to zero if negative. It should be noted that formula V requires that all epitopes have been haplotype mapped against identical sets of haplotypes.

Therefore, when selecting T-cell epitopes to be introduced in the analogue, it is important to include all knowledge of the epitopes which is available: 1) The frequency of responders in the population to each epitope, 2) MHC restriction data, and 3) frequency in the population of the relevant haplotypes.

There exist a number of naturally occurring “promiscuous” T-cell epitopes which are active in a large proportion of individuals of an animal species or an animal population and these are preferably introduced in the vaccine thereby reducing the need for a very large number of different analogues in the same vaccine.

The promiscuous (universal) epitope can according to the invention be a naturally occurring human T-cell epitope such as epitopes from tetanus toxoid (e.g. the P2 and P30 epitopes that are disclosed as part of constructs in WO 00/20027), diphtheria toxoid, Influenza virus hemagluttinin (HA), and P. falciparum CS antigen.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in analogues used according to the present invention. Cf. also the epitopes discussed in the following references which are hereby all incorporated by reference herein: WO 98/23635 (Frazer I H et al., assigned to The University of Queensland); Southwood S et. al, 1998, J. Immunol. 160: 3363-3373; Sinigaglia F et al., 1988, Nature 336: 778-780; Rammensee H G et al., 1995, Immunogenetics 41: 4 178-228; Chicz R M et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes which share common motifs with these.

Alternatively, the epitope can be any artificial T-cell epitope which is capable of binding a large proportion of haplotypes. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the modified antigen which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA or an immunologically effective subsequence thereof. This, and other epitopes having the same lack of MHC restriction are preferred T-cell epitopes which should be present in the analogues used in the inventive method. Such super-promiscuous epitopes will allow for the most simple embodiments of the invention wherein only one single analogue is presented to the vaccinated animal's immune system.

The nature of the above-discussed variation/modification preferably comprises that

at least one first moiety is included in the first and/or second analogue(s), said first moiety effecting targeting of the analogue to an antigen presenting cell (APC), and/or

at least one second moiety is included in the first and/or second analogue(s), said second moiety stimulating the immune system, and/or

at least one third moiety is included in the first and/or second analogue(s), said third moiety optimizing presentation of the analogue to the immune system.

The functional and structural features relating these first, second and third moieties will be discussed in the following:

They can be present in the form of side groups attached covalently or non-covalently to suitable chemical groups in the amino acid sequence of the polypeptide antigen or a subsequence thereof. This is to mean that stretches of amino acid residues derived from the polypeptide antigen are derivatized without altering the primary amino acid sequence, or at least without introducing changes in the peptide bonds between the individual amino acids in the chain.

The moieties can also be in the form of fusion partners to the amino acid sequence derived from the polypeptide antigen. In this connection it should be mentioned that both possibilities include the option of conjugating the amino acid sequence to a carrier, cf. the discussion of these below. In other words, in the present context the term “fusion protein is not merely restricted to a fusion construct prepared by means of expression of a DNA fragment encoding the construct but also to a conjugate between two proteins which are joined by means of a peptide bond in a subsequent chemical reaction.

As mentioned above, the analogue can also include the introduction of a first moiety which targets the analogue to an APC or a B-lymphocyte. For instance, the first moiety can be a specific binding partner for a B-lymphocyte specific surface antigen or for an APC specific surface antigen. Many such specific surface antigens are known in the art. For instance, the moiety can be a carbohydrate for which there is a receptor on the B-lymphocyte or the APC (e.g. mannan or mannose). Alternatively, the second moiety can be a hapten. Also an antibody fragment which specifically recognizes a surface molecule on APCs or lymphocytes can be used as a first moiety (the surface molecule can e.g. be an FCγ receptor of macrophages and monocytes, such as FCγRI or, alternatively any other specific surface marker such as CD40 or CTLA-4). It should be noted that all these exemplary targeting molecules can be used as part of an adjuvant, cf. below. CD40 ligand, antibodies against CD40, or variants thereof which bind CD40 will target the analogue to dendritic cells. At the same time, recent results have shown that the interaction with the CD40 molecule renders the T_{H} cells unessential for obtaining a CTL response. Hence, it is contemplated that the general use of CD40 binding molecules as the first moiety (or as adjuvants, cf. below) will enhance the CTL response considerably; in fact, the use of such CD40 binding molecules as adjuvants and “first moieties” in the meaning of the present invention is believed to be inventive in its own right.

As an alternative or supplement to targeting the analogue to a certain cell type in order to achieve an enhanced immune response, it is possible to increase the level of responsiveness of the immune system by including the above-mentioned second moiety which stimulates the immune system. Typical examples of such second moieties are cytokines, heat-shock proteins, and hormones, as well as effective parts thereof.

Suitable cytokines to be used according to the invention are those which will normally also function as adjuvants in a vaccine composition, e.g. interferon γ(IFN-γ), Flt3 ligand (Flt3L), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF); alternatively, the functional part of the cytokine molecule may suffice as the second moiety. With respect to the use of such cytokines as adjuvant substances. cf. the discussion below.

Alternatively, the second moiety can be a toxin, such as listeriolycin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities.

According to the invention, suitable heat shock proteins used as the second moiety can be HSP70, HSP90, HSC70, GRP94, and calreticulin (CRT).

Also the possibility of introducing a third moiety which enhances the presentation of the analogue to the immune system is an important embodiment of the invention. The art has shown several examples of this principle. For instance, it is known that the palmitoyl lipidation anchor in the Borrelia burgdorferi protein OspA can be utilised so as to provide self-adjuvating polypeptides (cf. e.g. WO 96/40718). It seems that the lipidated proteins form up micelle-like structures with a core consisting of the lipidation anchor parts of the polypeptides and the remaining parts of the molecule protruding therefrom, resulting in multiple presentations of the antigenic determinants. Hence, the use of this and related approaches using different lipidation anchors (e.g. a myristyl group, a farnesyl group, a geranyl-geranyl group, a GPI-anchor, and an N-acyl diglyceride group) are preferred embodiments of the invention, especially since the provision of such a lipidation anchor in a recombinantly produced protein is fairly straightforward and merely requires use of e.g. a naturally occurring signal sequence as a fusion partner for the analogue. Another possibility is use of the C3d fragment of complement factor C3 or C3 itself (cf. Dempsey et al., 1996, Science 271, 348-350 and Lou & Kohler, 1998, Nature Biotechnology 16, 458-462).

It is important to note that when attempting to use the method of the invention against e.g. membrane bound polypeptide antigens which are exposed to the extracellular compartment, it is most preferred that the first and/or second analogue(s) has/have substantially the overall tertiary structure of the polypeptide antigen. In the present specification and claims this is intended to mean that the overall tertiary structure of the part of the polypeptide antigen which is extracellularly exposed is preserved, since, as mentioned above, the tertiary structure of the obligate intracellular polypeptides do not engage the humeral immune system. In fact, as part of the vaccination strategy it is often desired to avoid exposure to the extracellular compartment of putative B-cell epitopes derived from intracellular part of the polypeptide antigens; in this way, potentially adverse effects caused by cross-reactivity with other antigens can be minimized.

For the purposes of the present invention, it is however sufficient if the variation/modification (be it an insertion, addition, deletion or substitution) gives rise to a foreign T-cell epitope and at the same time preserves a substantial number of the CTL epitopes in the polypeptide antigen (and sometimes also a substantial number of B-cell epitopes).

The following formula describes the constructs generally covered by the invention:

(MOD₁)ₛ₁(PAGₑ₁)ₙ₁ (MOD 2)ₛ₂(PAGₑ₂)ₙ₂ . . . . (MODₓ)ₛₓ(PAGₑₓ) (I)

where PAGₑ₁-PAGₑₓ are x CTL and/or B-Cell epitope containing subsequences of the relevant polypeptide antigen which independently are identical or non-identical and which may contain or not contain foreign side groups, x is an integer ≧3, n1−nx are x integers ≧0 (at least one is ≧1), MOD₁−MODₓ are x modifications introduced between the preserved epitopes, and s1−sx are x integers ≧0 (at least one is ≧1 if no side groups are introduced in the sequences). Thus, given the general functional restraints on the immunogenicity of the constructs, the invention allows for all kinds of permutations of the original antigen sequence, and all kinds of modifications therein. Thus, included in the invention are analogues obtained by omission of parts of the polypeptide antigen sequence which e.g. exhibit adverse effects in vivo or omission of parts which are normally intracellular and thus could give rise to undesired immunological reactions, cf. the detailed discussion below.

A further elaboration of the above principle include use of CTL and/or B-cell epitopes from more than one pathology-related antigen. For instance, there are several cancer related antigens that exert their oncogenic effects when they are in a mutated form only—examples are mutated K-ras and P53 which both are crucial proteins in normal cell cycle regulation and which both are expression products in most normal cells. In some cases, CTLs have been shown to recognise mutated peptides from these antigens. It is therefore important that the immune system responds to te mutated peptide only, and not to the unmutated parts, if antigen specific immunotherapy is instigated.

We have devised a strategy whereby sequences of 8-25 amino acids of such disease-related proteins could be used as further epitopes in an AutoVac construct—in preferred embodiments, the introduced epitopes would at the same time provide for the emergence of T_{H} epitopes in the final construct, cf. the discussion above. The epitopes used for this purpose would be those which comprise the mutated region of the disease-related protein. By using such an approach, it would be possible to generate CTLs (and possibly antibodies, where applicable) against only the mutated form of the disease-related antigen. In the cases where the disease-related antigen provides for the emergence of a T_{H} epitope, the use of a truly foreign T_{H} epitope could be completely omitted. An embodiment of this principle could e.g. be vaccination with a nucleic acid vaccine which encode an analogue of a polypeptide antigen (e.g. Her2 or PSM) wherein has been introduced at least one T_{H} epitope and at least one peptide derived from another disease-related antigen (e.g. a peptide from the mutated part of an oncogenic protein). In a preferred embodiment, the at least one T_{H} epitope is introduced as a consequence of the introduction of the peptide.

It is furthermore preferred that the variation and/or modification includes duplication, when applicable, of the at least one B-cell epitope, or of at least one CTL epitope of the polypeptide antigen. This strategy will give the result that multiple copies of preferred epitopic regions are presented to the immune system and thus maximizing the probability of an effective immune response. Hence, this embodiment of the invention utilises multiple presentations of epitopes derived from the polypeptide antigen (i.e. formula I wherein at least one B-cell epitope is present in two positions).

This effect can be achieved in various ways, e.g. by simply preparing fusion polypeptides comprising the structure (PAG)ₘ, where m is an integer ≧2 and then introduce the modifications discussed herein in at least one of the polypeptide antigen sequences.

An alternative embodiment of the invention which also results in the preferred presentation of multiple (e.g. at least 2) copies of the important epitopic regions of the antigen to the immune system is the covalent coupling of the antigen, subsequence or variants thereof to certain molecules. For instance, polymers can be used, e.g. carbohydrates such as dextran, cf. e.g. Lees A et al., 1994, Vaccine 12: 1160-1166; Lees A et al., 1990, J. Immunol. 145: 3594-3600, but also mannose and mannan are useful alternatives. Integral membrane proteins from e.g. E. coli and other bacteria are also useful conjugation partners. The traditional carrier molecules such as keyhole limpet haemocyanin (KLH), tetanus toxoid, diphtheria toxoid, and bovine serum albumin (BSA) are also preferred and useful conjugation partners.

Maintenance of the sometimes advantageous substantial fraction of B-cell epitopes or even the overall tertiary structure of a protein which is subjected to modification as described herein can be achieved in several ways. One is simply to prepare a polyclonal antiserum directed against the polypeptide antigen (e.g an antiserum prepared in a rabbit) and thereafter use this antiserum as a test reagent (e.g. in a competitive ELISA) against the modified proteins which are produced. Modified versions (analogues) which react to the same extent with the antiserum as does the polypeptide antigen must be regarded as having the same overall tertiary structure as the polypeptide antigen whereas analogues exhibiting a limited (but still significant and specific) reactivity with such an antiserum are regarded as having maintained a substantial fraction of the original B-cell epitopes.

Alternatively, a selection of monoclonal antibodies reactive with distinct epitopes on the polypeptide antigen can be prepared and used as a test panel. This approach has the advantage of allowing 1) an epitope mapping of the polypeptide antigen in question and 2) a mapping of the epitopes which are maintained in the analogues prepared.

Of course, a third approach would be to resolve the 3-dimensional structure of the polypeptide antigen or of a biologically active truncate thereof (cf. above) and compare this to the resolved three-dimensional structure of the analogues prepared. Three-dimensional structure can be resolved by the aid of X-ray diffraction studies and NMR-spectroscopy. Further information relating to the tertiary structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the tertiary structure of a given molecule. However, ultimately X-ray diffraction and/or NMR are necessary to obtain conclusive data since circular dichroism can only provide indirect evidence of correct 3-dimensional structure via information of secondary structure elements.

For the purposes of the present invention it should also be mentioned that instead of using naturally occurring B and CTL epitopes, it is possible to use mimotopes which are isolated from e.g. phage libraries which are tested against antibodies which are known to bind to the relevant antigen.

In essence there are at present three feasible ways of obtaining the presentation of the relevant epitopes to the immune system: Traditional sub-unit vaccination with polypeptide antigens, administration of a genetically modified live vaccine, and nucleic acid vaccination. These three possibilities will be discussed separately in the following:

Polypeptide Vaccination

This entails administration to the animal in question of an immunogenically effective amount of the polypeptide antigen or the variant thereof, and, when relevant, administration of an immunologically effective amount of the at least one second analogue.

The chitosan formulation of polypeptides can be accomplished in a number of ways. Simple admixture of chitosan and polypeptide is one way, but in preferred embodiments of the invention, the polypeptide is part of a chitosan microparticle (i.e. a bead, microsphere or microcapsule). Methods for preparing and loading such microparticles are well-known in the art, cf. e.g Kas H S, 1997, J. Microencapsulation 14(6), 689-711.

The mean molecular weight of the chitosan is of importance for the size of the microparticles produced but may also have implications for the immunogenic properties of simple admixtures of chitosan and polypeptide antigen. Generally, the chitosan should have a mean molecular weight in the range from about 3,000 to about 3,000,000, where the preferred molecular weight is in the range from about 30,000 to about 2,000,000. Especially preferred are molecular weights in the range from about 50,000 to about 500,000, and more preferred are molecular weights in the range from about 60,000 to about 400,000. Still more preferred are molecular weights in the range from about 70,000 to about 300,000 and even more preferred are molecular weights in the range from about 80,000 to about 200,000. Most preferred are molecular weights in the range from about 90,000 to about 150,000, notably molecular weights ranging from about 95,000 to about 130,000.

Also the viscosity of the chitosan molecules, as measured for 1% chitosan in 1% acetic acid, are of importance. Generally, viscosities will preferably range from about 2 mPas to about 500 mPas, with preferred viscosities in the range from about 3 to about 300 mPas. Especially preferred are viscosities in the range from about 4 to about 200 mPas, and even more preferred are viscosities in the range from about 5 to about 150 mPas. Yet more preferred are viscosities in the range from about 6 to about 120 mPas, with especially preferred viscosities in the range from about 7 to about 100 mPas. Most preferred are viscosities in the range from about 8 to about 80 mPas with very useful viscosities in the range from about 9 to about 60 mPas. Especially good results and particle sizes are obtained when using chitosan viscosities in the range from about 10 to about 40 mPas, where viscosities in the range from about 11 to about 20 mPas are most preferred. The examples given herein have utilised chitosan with a viscosity of about 12 mPas.

A third characteristic of the chitosan molecules which is of importance is their degree of acetylation (measured as the percentage of acetyl groups in the chitosan molecule relative to the maximum possible number of acetyl groups of a corresponding chitin molecule)—normally, this characteristic is expressed as the degree of deacetylation, i.e. 100% minus the percentage of acetylation. The degree of deacetylation is of importance because it e.g. determines the net charge of the chitosan particles prior to loading with antigen.

In general, the degrees of deacetylation of the chitosan molecules are those of at least about 65%, but in general it is preferred that the degree of deacetylation is as high as possible. Therefore, it is preferred that the deacetylation degree is at least 70%, such as at least 75%, but even higher degrees of deacetylation are preferred such as at least 80% or higher, e.g. at least 85%. Most preferred are degrees of deacetylation of at least 87%, more preferred at least 89% and even more preferred at least 91%. Very good results are expected if using chitosan having a degree of deacetylation of at least 93%, such as at least 95%, or even at least 97%. As shown in the examples, the use of chitosan with a deacetylation degree of more than 98% have proven very effective, and this is thus the most preferred embodiment.

The mean diameter of chitosan microparticles should be in the range between 0.1 and 10 um, preferably between 0.2 and 5 μm, more preferred between 0.3 and 2.5 μm, especially in the range between 0.4 and 2 um, and most preferred between 0.5 and 1.5 μm. Very good results are expected when the particle diameter is between 0.6 and 1.3 μm, such as between 0.65 and 1.2 μm, especially between 0.7 and 1.0 μm. Most preferred, the particle diameter should be in the range between 0.73 and 0.82 μm.

Finally, the mean ζ (zeta) potential of the unloaded microparticles should in general be in the range from about +0.5 to about +50 mV, with preferred mean ζ potentials in the upper part of this range. Thus, preferred mean ζ potentials range from about 15 to about 45 mV, with more preferred mean ζ potentials in the range from about 20 to about 42 mV. Most preferred are mean ζ potentials in the range between 25 and 41 mV, such as between 30 and 40 mV, and more precisely in the range between 33 and 39 mV, such as between 34 and 38 mV.

It has proven effective to stabilise the chitosan particles characterized above by subsequent cross-linking. The crosslinking may be performed by any suitable method known in the art, i.e. by means of cross-linking agents such as glutaraldehyde and formaldehyde or by means of gelling agents such as alginate. The cross-linking of the chitosan particles may be performed both prior to loading with immunogen or after, according to the methods known in the art referred to in the following.

In general, the polypeptide antigen or variant thereof is included in the particles by means of passive absorption or any other suitable method of incorporation so as to obtain loaded particles with a suitable ratio between chitosan and polypeptide. This ratio should normally be in the range between 10 and 1 (measured as g chitosan per 9 polypeptide), with preferred ratios in the range between 8 and 1.3, such as the range between 5 and 1.5. Most preferred are ratios between 3 and 1.7, such as ratios between 2.5 and 1.8. It is most preferred that the ratio is about 2.

It is believed that the inclusion of minor amounts of detergent in the chitosan formulation will enhance the CTL induction mediated by immunogenic composition. Without being limited to any theory, it is nevertheless believed that the chitosan facilitate pinocytotic uptake by APCs of the polypeptide antigen or the variant thereof. After entry into the endosomal compartment the chitosan is degraded, and the detergent may thereafter facilitate the release of the polypeptide from the endosomes.

It will be clear for the skilled artisan that the amount and activity of the detergent must be adjusted carefully so as to on the one hand facilitate CTL responses while on the other hand avoiding toxic side effects exerted on the APC. Or, in other words, the amount of detergent must be effective but on the other hand pharmaceutically acceptable.

The detergent can be any one of the adjuvants described herein which are at the same time detergents, cf. below. Very good candidates are QuilA, listeriolysin, Tween 20, and Tween 80, but the choice of detergent is not critical.

The amount of detergent will have to be determined, depending on the precise choice thereof. The easiest way to determine a useful amount is to admix chitosan or to load microparticles with varying amounts of detergent and immunogenic polypeptide, and thereafter determine the detergent concentration which results in the highest degree of the desired immunologic reactivity.

It should be noted that since the presently used method for preparation of microparticles entail the use of the detergent Tween 80, it is believed that residual amounts of this detergent may be responsible for the high CTL induction and specific T-cell proliferation seen after administration to mice.

As an alternative to formulations of polypeptide antigen or variant thereof in chitosan, one may advantageously couple the polypeptide antigens or variants of the immunogenic composition covalently to chitosan, e.g. by standard methods for chemical conjugation of molecules.

Preferably, the polypeptide antigen or variant thereof (e.g. the at least one first and/or second analogue(s)) is/are further formulated together with a pharmaceutically and immunologically acceptable carrier and/or vehicle and, optionally an adjuvant.

When effecting presentation of the analogue to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intradermally, subdermally or intramuscularly. Additional formulations which are suitable for other modes of administration include suppositories and, in some cases, oral, buccal, sublinqual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 12%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The polypeptides may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to '500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the polypeptides of the vaccine are sufficiently immunogenic in a vaccine, but for some of the others the immune response will be enhanced if the vaccine further comprises an adjuvant substance. It is especially preferred to use an adjuvant which can be demonstrated to facilitate breaking of the autotolerance to autoantigens.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generationn Immunological Adjuvants”, 1995, Gregoriadis G et al. (eds.), Plenum Press, New York, ISBN 0-306-45283-9, both of which are hereby incorporated by reference herein.

Preferred adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the analogues also refer mutates mutandis to their use in the adjuvant of a vaccine of the invention.

The application of adjuvants include use of agents such as aluminum hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and ?-inulin, but also Freund's complete and incomplete adjuvants as well as quillaja saponins such as QuilA and QS21 are interesting. Further possibilities are monophosphoryl lipid A (MPL), and the above mentioned C3 and C3d.

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992, J. Immunol 149(11): 3477-81 (which is hereby incorporated by reference herein). In brief, the presentation of a relevant antigen such as an antigen of the present invention can be enhanced by conjugating the antigen to antibodies (or antigen binding antibody fragments) against the Fcγ receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned above as candidates for the first and second moieties in the modified analogues. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structrue and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12^{th}-5^{th}1998, Seascape Resort, Aptos, California”.

Recent findings have demonstrated that the co-administration of H2 agonists enhances the in-tumour survival of Natural Killer Cells and CTLs. Hence, it is also contemplated to include H2 agonists as adjuvants in the methods of the invention.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefor the immune system needs to be periodically challenged with the analogues.

It should be noted that vaccination with peptides/polypeptides according to the present invention may be the second step (“primary boost”) which follows after nucleic acid vaccination. Extremely promising results have been obtained using such an immunization strategy with Her2 antigen variants described herein, even without using chitosan in the formulation but by otherwise following the teaching of the present invention.

Due to genetic variation, different individuals may react with immune responses of varying strength to the same polypeptide. Therefore, the vaccine according to the invention may comprise several different polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different modified or unmodified polypeptides, such as 3-10 different polypeptides. However, normally the number of peptides will be sought kept to a minimum such as 1 or 2 peptides.Nucleic acid vaccination

As an alternative to classic administration of a peptide-based vaccine, the technology of nucleic acid vaccination (also known as “nucleic acid immunisation”, “genetic immunisation”, “gene immunisation” and “DNA vaccination) offers a number of attractive features. Hence, although the main focus of the present invention is polypeptide vaccination, it is believed that certain of the present chitosan formulations described above may prove superior to existing nucleic acid vaccine formulations.

First, in contrast to the traditional vaccine approach, nucleic acid vaccination does not require resource consuming large-scale production of the immunogenic agent (e.g. in the form of industrial scale fermentation of microorganisms producing the wild-type polypeptides or the analogues necessary in polypeptide vaccination). Furthermore, there is no need to device purification and refolding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original B-cell epitopes should be preserved in the analogues derived from extracellularly exposed polypeptide sequences, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is best ensured by having the host producing the immunogen.

Hence, an important embodiment of the method of the invention involves that presentation is effected by in vivo introducing, into the APC, at least one nucleic acid fragment which encodes and expresses the at least one CTL epitope and/or the at least one B-cell epitope, and the at least one first foreign T_{H} epitope (an alternative encompasses administration of at least 2 distinct nucleic acid fragments, where one encodes the at least one CTL epitope and the other encodes the at least one foreign T_{H} epitope). Preferably, this is done by using a nucleic acid fragment which encodes and expresses the above-discussed first analogue. If the first analogue is equipped with the above-detailed T_{H} epitopes and/or first and/or second and/or third moieties, these are then present in the form of fusion partners to the amino acid sequence derived from the polypeptide antigen, the fusion construct being encoded by the nucleic acid fragment.

As for the traditional vaccination approach, the nucleic acid vaccination can be combined with in vivo introduction, into the APC, of at least one nucleic acid fragment encoding and expressing the second analogue. The considerations pertaining to 1^{st}, 2^{nd} and 3^{rd} moieties and T_{H} epitopes apply also here.

In this embodiment, the introduced nucleic acid is preferably DNA which can be in the form of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology. The same holds true for other considerations relating to formulation and mode and route of administration and, hence, also these considerations discussed above in connection with a traditional vaccine apply mutatis mutandis to their use in nucleic acid vaccination technology.

One especially preferred type of formulation of nucleic acid vaccines are microparticles containing the DNA. Suitable microparticles are e.g. described in WO 98/31398. Further, also here the teachings relating to the use of chitosan formulations as described above apply mutatis mutandis.

Furthermore, the nucleic acid(s) used as an immunization agent can contain regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different open reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Under normal circumstances, the nucleic acid of the vaccine is introduced in the form of a vector wherein expression is under control of a viral promoter. For more detailed discussions of vectors according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

It should be noted that preferred analogues used in the methods of the invention comprise modifications which results in a polypeptide having a sequence identity of at least 70% with the polypeptide antigen or with a subsequence thereof of at least 10 amino acids in length. Higher sequence identities are preferred, e.g. at least 75% or even at least 80% or 85%. The sequence identity for proteins and nucleic acids can be calculated as (N_{ref}-N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8)

Antigens and Tools Used in the Invention

Details concerning specific variants of antigens as well as biochemical and biological tools used for their production is discussed in detail in WO 00/20027, which is hereby incorporated by reference herein. All disclosures in WO 00/20027 pertaining to any of the weak polypeptide antigens mentioned herein pertain equally for the purposes of the present invention—this also includes the specific rationale for choosing these antigens as targets for the presently claimed therapeutic vaccination method. On pages 55 through 65, WO 00/20027 lists these antigens; the application of the present invention on any one of these antigens is an especially preferred embodiment of the present invention. However, as mentioned above, the present invention is applicable to any antigen towards which it would be desired to raise a CTL response.

4 of the antigens listed in WO 00/20027 are of special interest, namely PSM, Her2, hCG and FGF8b. These, and all pertinent antigens in the above-referenced table will eventually be formulated according to the principles of the present invention.

Hence, the method of the invention preferably entails that a foreign T_{H}-cell epitope is introduced in a part of the PSM amino acid sequence defined by SEQ ID NO: 2 positions 16-52 and/or 87-108 and/or 210-230 and/or 269-289 and/or 298-324 and/or 442-465 and/or 488-514 and/or 598-630 and/or 643-662 and/or 672-699. Furthermore, a modified PSM molecule which has a foreign T_{H}-epitope introduced in these positions is also a part of the invention.

A further important embodiment of the methods of the invention is one wherein the foreign T-cell epitope is introduced in a part of the Her2 amino acid sequence defined by the amino acid numbering in SEQ ID NO: 3 positions 5-25 and/or 59-73 and/or 103-117 and/or 149-163 and/or 210-224 and/or 250-264 and/or 325-339 and/or 369-383 and/or 465-479 and/or 579-593 and/or 632-652 and/or 653-667 and/or 661-675 and/or 695-709 and/or 710-730, cf. the Examples.

Finally, the invention also relates to embodiments of the methods described herein where, where the foreign T-cell epitope is introduced in a part of the FGF8b amino acid sequence defined by SEQ ID NO: 6 positions 1-54 and/or 178-215 and/or 55-58 and/or 63-68 and/or 72-76 and/or 85-91 and/or 95-102 and/or 106-111 and/or 115-120 and/or 128-134 and/or 138-144 and/or 149-154 and/or 158-162 and/or 173-177. It should be noted that it is especially preferred not to introduce variations or modifications in positions 26-45 and in the C-terminus starting at amino acids 186-215, since these stretches show the least homology with a recently discovered protein, FGF-18, which seems to be expressed in a variety of non-tumour tissues.

Furthermore, the present invention also pertains to methods of the invention employing specifically modified versions of the human mucins, especially any of MUC-1 through MUC-4, preferably MUC-1. The analogues comprise the following structure

TR (-S-P-S-(TR)ₘ)ₙ

where TR is a tandem repeat derived from the naturally occurring mucin, P is a foreign T_{H}-epitope as discussed herein, S is an inert spacer peptide having from 0 to 15 amino acid residues, preferably between 0 and 10 amino acid residues, and n is an integer of from 1 to 30, and m is an integer from 1 to 10, preferably from 3 to 5.

Compositions of the Invention

The invention also relates to an immunogenic composition which comprises chitosan as described above and, as a specific part of the immunogenic agent, at least one of the polypeptides or nucleic acid fragments described herein in admixture with a pharmaceutically and immunologically acceptable carrier, vehicle, diluent, or excipient, and optionally an adjuvant, cf also the discussion of these entities in the description of the method of the invention above.

### EXAMPLE 1

Preparation of Chitosan Microparticles

0.2 g of chitosan base (ChitoClear™ 804 from Primex Ingredients, Viscosity: 12 mPas measured in 1% in 1% acetic acid, deacetylation: 98.3%) and 0.8 g of Tween 80 is weighed out in a 100 ml beaker and brought into solution by addition of 80 ml of 2% acetic acid and subsequent stirring so as to obtain a solution of 0.25% chitosan, 1% Tween 80 and 2% acetic acid.

The beaker is placed in an ultrasound probe device (Soniprep 150, MSE) with a magnet stirring device. The solution is sonicated with a small probe for 30 min at 6 mA and magnetic stirring. Initially, sodium sulphate solution is added dropwise until particles precipitate (the amount and concentration can vary, e.g. 2 ml 10% sodium sulphate, 1 ml 20% sulphate etc.).

The particles are spun down in two 50 ml tubes at 5000 rpm for 20 min (Stratos Biofuge, Heraeus Instruments). The supernatant is isolated and resuspended in MilliQ water. Each batch is pooled in a tube and water is added up to approximately 35 ml. The tubes are centrifuged again. This wash procedure is repeated two more times.

After the 3^{rd} wash, 30 ml MilliQ water is added and the centrifugation is performed at 500 rpm for 10 min in order to remove ultrasound “metal dust”. The centrifugation is repeated. The Supernatant is transferred to new 50 ml tubes rd before centrifuging a 3 time.

A 250 flask is weighed and the mass is noted. 1.5 trehalose dihydrate is weighed (precisely) out in the flask, corresponding to 1.5×342.3/378.3 g trehalose. Thereafter, the 30 ml suspension containing chitosan microparticles is added. The trehalose must be solubilized. The resulting mixture is freeze dryed by means of ethanol and dry ice.

The following day, the flask is weighed to allow for the calculation of the amount of particles (Second weight of flask minus first weight of flask and weight of trehalose).

### EXAMPLE 2

Loading of Chitosan Microparticles with Ovalbumin

Solutions of 20 mg/ml chitosan particles in water are prepared as well as solutions of 20 mg/ml ovalbumin in water. 0.5 ml of each solution are mixed in an Eppendorf tube which is left to incubate for 3 hours at room temperature.

After 3 hours, the suspension is transferred to a 10 ml tube and 4 ml MilliQ is added. The resulting mixture is centrifuged at 10,000 rpm for 15 min. The supernatant is removed by suction and the pellet is resuspended in 5 ml MilliQ water. The mixture is centrifuged again. This procedure is repeated 3 times. The amount of ovalbumin in the supernatant (i.e. non-bound ovalbumin) is determined by means of a BCA assay:

A standard solution of ovalbumin in water containing 0.5 mg/ml is prepared. This standard is diluted to 0.4, 0.2, 0.1, 0.05 and 0.0125 mg/ml. 20 μl per well of each of these 7 standards as well as a blind are added in triplicate to a flat-bottomed microtiter plate, cf. the scheme below. The supernatants (some in diluted form, cf. the scheme below) from the loading are added at 20 μl per well.

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | |
|---|---|---|
| A | 0.5 mg/ml OVA | 1^{st} sup. dil. x2 |
| B | 0.4 mg/ml OVA | 1^{st} sup. dil. x4 |
| C | 0.2 mg/ml OVA | 1^{st} sup. dil. x8 |
| D | 0.1 mg/ml OVA | 2^{nd} sup. dil. x1 |
| E | 0.05 mg/ml OVA | 3^{rd} sup. dil. x1 |
| F | 0.025 mg/ml OVA | |
| G | 0.0125 mg/ml OVA | |
| H | 0 mg/ml OVA | |

200 μl BCA reagent (1 part cupper(II) sulphate pentahydrate 4. to 50 parts bicinchoninic acid solution) is added to all wells.

The plate is incubated at approximately 50° C. for 30-45 min. After cooling to room temperatures the plate is assayed spectrophotometrically at 562 nm.

### EXAMPLE 3

Assaying of CTL Induction and T-Cell Proliferation

Mice have been injected subcutaneously with the following:

1. 200 μl ovalbumin-loaded (0.5 μg/ml) chitosan particles prepared as above (10 μg chitosan per 5 μg ovalbumin).

2. 200 μl ovalbumin/chitosan mixture (0.5 μg/ml ovalbumin and approximately same ratio between chitosan and ovalbumin).

3. 200 μl ovalbumin in Freund's complete adjuvant (0.5 μg/ml ovalbumin).

4. 200 ul of the peptide SIINFEKL (a known CTL epitope from ovalbumin) in Freund's complete adjuvant (0.5 μg/ml SIINFEKL).

5. 200 μl ovalbumin in H₂O (0.5 μg/ml ovalbumin).

6. 200 μl H₂O.

Ten days after last immunization, the mice were sacrificed and axillar and inguinal lymph nodes and the spleens were excised.

In a standard Chrome release assay for determination of CTLs lysing SIINFEKL-carrying cells, results have been obtained showing that CTLs were induced by the albumin-loaded chitosan particles to the same degree as both Ova in FCA and SIINFEKL in FCA.

Further, in a standard proliferation assay where the specific reactivity of T-cells is gauged, it was shown that the immunization with ovalbumin-loaded chitosan particles results in a proliferation index which is more than 2×higher than that obtained using ovalbumin in FCA, meaning that the chitosan formulation exhibits a superior capability of inducing antigen-specific T-cell proliferation.

### EXAMPLE 4

Cross-linking of chitosan sulphate microparticles

In order to obtain a chitosan microparticle formulation with increased stability, the following cross-linking procedure is applied. 25 μl of an aqueous glutaraldehyde solution (25%) is added at 25° C. at 1000 rpm (Thermomixer compact, Eppendorf, Hamburg, Germany) to 1 ml of microparticle suspension (0.5% w/v) as prepared above in EXAMPLE 1.

The cross-linking reaction is stopped after 5 minutes by adding 30% (v/v) hydrogen peroxide solution (four times the amount of glutaraldehyde).

The cross-linked microparticles are subsequently purified by 3 rounds of centrifugation+resuspension in water.

The microparticle suspension can be non-loaded or protein-loaded microparticles. Cross-linking time and cross-linking agent amount can be varied: Equally good results are obtained when using 10 >1 of glutaraldehyde and stopping the crosslinking after 1 hour.

Stability studies have shown that the microparticles obtained by means of this procedure are more stable than the particles obtained directly from Example 1. Currently, immunization experiments corresponding to those of Example 3 are performed with the cross-linked chitosan formulations in order to confirm that they will also be effective in inducing CTL responses.

1. A method for inducing or enhancing an immune response against a polypeptide antigen in an animal, including a human being, said polypeptide antigen being weakly immunogenic or non-immunogenic in the animal, the method comprising administering, to the animal, the polypeptide antigen or at least one variant thereof which includes at least one first T-helper Cell epitope that is foreign to the animal (foreign T_{H} epitope), wherein the polypeptide antigen or variant thereof is formulated with chitosan.

2. The method of claim 1, which comprises effecting simultaneous presentation by antigen presenting cells (APCs) of the animal's immune system of an immunogenically effective amount of
1) at least one CTL epitope derived from the polypeptide antigen, and
2) the at least one first foreign T_{H} epitope.

3. The method according to claim 2 wherein the polypeptide antigen is a cell-associated polypeptide antigen and wherein the method includes down-regulating the cell-associated polypeptide antigen in the animal by inducing a specific cytotoxic T-lymphocyte (CTL) response against cells carrying the cell-associated polypeptide antigen on their surface or harbouring the cell-associated polypeptide antigen in their intracellular compartment, the method comprising effecting, in the animal, simultaneous presentation by a suitable antigen presenting cell (APC) of
1) at least one CTL epitope derived from the polypeptide antigen, and
2) the at least one first T_{H} epitope.

4. The method according to claim 2 or 3, wherein said at least one CTL epitope when presented is associated with an MHC Class I molecule on the surface of the APC and/or wherein said at least one first foreign T_{H} epitope when presented is associated with an MHC Class II molecule on the surface of the APC.

5. The method according to any one of the preceding claims, wherein the APC is a dendritic cell or a macrophage.

6. The method according to any one of the preceding claims, wherein the polypeptide antigen is selected from a tumour-associated polypeptide antigen, a self-protein, a viral polypeptide antigen, and a polypeptide antigen derived from an intracellular parasite or bacterium.

7. The method according to any one of claims 2-6, wherein presentation by the APC of the CTL epitope and the first foreign T_{H} epitope is effected by presenting the animal's immune system with the variant in the form of at least one first analogue of the polypeptide antigen, said first analogue comprising a variation of the amino acid sequence of the polypeptide antigen, said variation containing at least the CTL epitope and the first foreign T_{H} epitope.

8. The method according to claim 7, wherein the variation and/or modification involves amino acid substitution and/or deletion and/or insertion and/or addition.

9. The method according to claim 7 or 8, wherein the variation and/or modification comprises that
at least one first moiety is included in the first analogue, said first moiety effecting targeting of the analogue to an antigen presenting cell (APC), and/or
at least one second moiety is included in the first analogue, said second moiety stimulating the immune system, and/or
at least one third moiety is included in the first analogue, said third moiety optimizing presentation of the analogue to the immune system.

10. The method according to any one of the preceding claims, wherein the first foreign T_{H} epitope is immunodominant and/or wherein the first foreign T_{H} epitope is promiscuous.

11. The method according to any one of the preceding claims, wherein the first foreign T_{H} epitope is selected from a natural T_{H} epitope and an artificial MHC-II binding peptide sequence.

12. The method according to claim 11, wherein the natural T_{H} epitope is selected from a Tetanus toxoid epitope such as P2 or P30, a diphtheria toxoid epitope, an influenza virus hemagluttinin epitope, and a P. falciparum CS epitope.

13. The method according to any one of the preceding claims, wherein the first T_{H} epitope and/or first and/or second and/or third moieties are present in the form of
side groups attached covalently or non-covalently to suitable chemical groups in the amino acid sequence of the polypeptide antigen or a subsequence thereof, and/or
fusion partners to the amino acid sequence derived from the polypeptide antigen.

14. The method according to claim 13, wherein the first moiety is a substantially specific binding partner for an APC specific surface antigen such as a carbohydrate for which there is a receptor on the APC, e.g. mannan or mannose, wherein the second moiety is a cytokine selected from interferon y (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-1S), and granulocyte-macrophage colony stimulating factor (GM-CSF), or an effective part thereof; a heat-shock protein selected from HSP70, HSP90, HSC70, GRP94, and calreticulin (CRT), or an effective part thereof; or a hormone, and wherein the third moiety is a lipid such as a palmitoyl group, a myristyl group, a farnesyl group, a geranyl-geranyl group, a GPI-anchor, and an N-acyl diglyceride group.

15. The method according to any one of the preceding claims, wherein the polypeptide antigen or the variant in addition to chitosan is formulated together with a pharmaceutically and immunologically acceptable carrier and/or vehicle and, optionally an adjuvant.

16. The method according to claim 15, wherein said adjuvant facilitates uptake by APCs, such as dendritic cells, of the polypeptide antigen or variant.

17. The method according to claim 16, wherein the adjuvant is selected from the group consisting of an immune targeting adjuvant; an immune modulating adjuvant such as a toxin, a cytokine, and a mycobacterial derivative; an oil formulation;
a polymer; a micelle forming adjuvant; a saponin; an immunostimulating complex matrix (ISCOM matrix); a particle;
DDA; aluminium adjuvants; DNA adjuvants; ?-inulin; and an encapsulating adjuvant.

18. The method according to any one of the preceding claims, which includes administration via a route selected from the oral route and the parenteral route such as the intradermal, the subdermal, the intracutaneous, the subcutaneous; the peritoneal, the buccal, the sublinqual, the epidural, the spinal, the anal, and the intracranial routes.

19. The method according to any one of the preceding claims, which includes at least one administration a year, such as at least 2, 3, 4, 5, 6, and 12 administrations a year.

20. The method according to any one of the preceding claims, wherein the weak cell-associated antigen is selected from the group consisting of 5 alpha reductase, a-fetoprotein, AM-1, APC, APRIL, BAGE, β-catenin, Bcl2, bcr-abl (b3a2), CA-125, CASP-8/FLICE, Cathepsins, CD19, CD20, CD21, CD23, CD22, CD33, CD35, CD44, CD45, CD46, CD5, CD52, CD55 (791Tgp72), CD59, CDC27, CDK4, CEA, c-myc, Cox-2, DCC, DcR3, E6/E7, EGFR, EMBP, Ena78, farsyl transferase, FGF8a or FGF8b, FLK1/KDR, Folic Acid Receptor, G250, GAGE-Family, gastrin 17, Gastrin-releasing hormone (Bombesin), GD2/GD3/GM2, GnRH, GnTV, GP1, gp100/Pmel 17, gp-100-in4, gp15, gp75/TRP-1, hCG, Heparanase, Her2/neu, HMTV, Hsp70, hTERT (telomerase), IGFR1, IL-13R, iNOS, Ki 67, KIAA0205, K-ras, H-ras, N-ras, KSA (CO17-lA), LDLR-FUT, MAGE Family (MAGE-1, MAGE-2, MAGE-3, etc), Mammaglobin, MAP17, Melan-A/MART-1, mesothelin, MIC A/B, MT-MMP's, Mox1, Mucin such as MUC-1, MUC-2, MUC-3, and MUC-4 being abberantly glycosylated, MUM-1, NY-ESO-1, Osteonectin, p15, P170/MDR1, p53, p97/melanotransferrin, PAI-1, PDGF, Plasminogen (uPA), PRAME, Probasin, Progenipoietin, PSA, PSM, RAGE-1, Rb, RCAS1, SART-1, SSX gene family, STAT3, STn (mucin assoc.), TAG-72, TGF-a, TGF-β, Thymosin β 15, TNF-a, TPA, TPI, TRP-2, Tyrosinase, VEGF, ZAG, p161NK4, and Glutathione S-transferase.

21. The method according to claim 20, wherein the cell-associated polypeptide antigen is human PSM.

22. The method according to claim 21, wherein the foreign T-cell epitope is introduced in a part of the PSM amino acid sequence defined by SEQ ID NO: 2 positions 16-52 and/or 87-108 and/or 210-230 and/or 269-289 and/or 298-324 and/or 442-465 and/or 488-514 and/or 598-630 and/or 643-662 and/or 672-699.

23. The method according to claim 21 or 22 used in the treatment or amelioration of prostate cancer.

24. The method according to claim 20, wherein the cell-associated polypeptide antigen is fibroblast growth factor 8b (FGF8b).

25. The method according to claim 24, where the foreign T-cell epitope is introduced in a part of the FGF8b amino acid sequence defined by SEQ ID NO: 6 positions 1-54 and/or 178-215 and/or 55-58 and/or 63-68 and/or 72-76 and/or 85-91 and/or 95-102 and/or 106-111 and/or 115-120 and/or 128-134 and/or 138-144 and/or 149-154 and/or 158-162 and/or 173-177, and wherein the introduction preferably does not substantially involve amino acids 26-45 and amino acids 186-215.

26. The method according to claim 24 or 25 used in the treatment or amelioration of cancer such as prostate cancer and breast cancer.

27. The method according to claim 20, wherein the cell-associated polypeptide antigen is Her2.

28. The method according to claim 27, wherein the foreign T-cell epitope is introduced in a part of the Her2 amino acid sequence defined by SEQ ID NO: 3 positions 5-25 and/or 59-73 and/or 103-117 and/or 149-163 and/or 210-224 and/or 250-264 and/or 325-339 and/or 369-383 and/or 465-479 and/or 579-593 and/or 632-652 and/or 653-667 and/or 661-675 and/or 695-709 and/or 710-730.

29. The method according to claim 27 or 28 used in the treatment or amelioration of breast cancer.

30. The method according to any one of the preceding claims, wherein the polypeptide antigen or the variant thereof is part of a simple mixture with chitosan.

31. The method according to any of claims 1-29, wherein the polypeptide antigen or variant thereof is formulated in a chitosan microparticle, such as a bead, microsphere or microcapsule.

32. The method according to claim 30 or 31, wherein the mean molecular weight of the chitosan molecules used for preparation of the formulation is in the range from about 3,000 to about 3,000,000, preferably in the range from about 30,000 to about 2,000,000, more preferred in the range from about 50,000 to about 500,000, even more preferred in the range from about 60,000 to about 400,000, still more preferred in the range from about 70,000 to about 300,000, especially preferred in the range from about 80,000 to about 200,000.

33. The method according to claim 32, wherein the mean molecular weight is in the range from about 90,000 to about 150,000, preferably from about 95,000 to about 130,000

34. The method according to any of claims 30-33, wherein the chitosan used for preparing the formulation has a viscosity, as measured for 1% chitosan in 1% acetic acid, range from about 2 mPas to about 500 mPas, preferably in the range from about 3 to about 300 mPas, more preferred from about 4 to about 200 mPas, even more preferred from about 5 to about 150 mPas, still more preferred from about 6 to about 120 mPas, and even still more preferred from about 7 to about 100 mPas.

35. The method according to claim 34, wherein the viscosity is in the range from about 8 to about 80 mPas, preferably from about 9 to about 60 mPas, even more prefeably from about 10 to about 40 mPas, still more preferably from about 11 to about 20 mPas, and especially in a range close around 12 mPas.

36. The method according to any one of claims 30-35, wherein chitosan used for preparing the formulation has a degree of deacetylation of at least 65%, such as at least 70%, at least 75%, at least 80%, and at least 85%, preferably at least 87%, such as at least 89%, at least 91%, and at least 93%, and more preferred at least 95%, such as at least 97%, and most preferred at least 98%.

37. The method according to any of claims 31 and 32-36 insofar as these are depending on claim 31, wherein the mean diameter of chitosan microparticles is in the range from about 0.1 to about 10 μm, preferably between 0.2 and 5 μm, more preferred between 0.3 and 2.5 μm, especially preferred in the range between 0.4 and 2 μm, and most preferred between 0.5 and 1.5 μm.

38. The method according to claim 37, wherein the mean particle diameter is between 0.6 and 1.3 μm, such as between 0.65 and 1.2 μm, especially between 0.7 and 1.0 μm, and preferably between 0.73 and 0.82 μm.

39. The method according to any of claims 31 and 32-38 insofar as these depend on claim 60, wherein the mean ? potential of unloaded chitosan microparticles is in the range from about +0.5 to about +50 mV, such as from about 15 to about 45 mV, from about 20 to about 42 mV, from about 25 to about 41 mV, from about 30 to about 40 mV, from about 33 to about 39 mV, and from about 34 to about 38 mV.

40. The method according to any one of claims 30-39, wherein the ratio (w/w) between chitosan and polypeptide or nucleic acid is in the range between 10 and 1, such as between 8 and 1.3, between 5 and 1.5, between 3 and 1.7, between 2.5 and 1.8, and preferably the ratio is about 2.

41. The method according to any one of claims 30-40, which also comprises a pharmaceutically acceptable amount of a detergent, such as QuilA, listeriolysin, Tween 80 or Tween 20.

42. An immunogenic composition which comprises chitosan in admixture with 1) a polypeptide antigen or variant as these are defined in any one of claims 6-14, 20-22, 24, 25, 27 and 28 and 2) a pharmaceutically and immunologically acceptable carrier or vehicle, and optionally a further adjuvant.

43. The immunogenic composition according to claim 42, wherein the chitosan component is as defined in any one of claims 3040.

44. The immunogenic composition according to claim 42 or 43, further comprising a pharmaceutically acceptable amount of a detergent, such as QuilA, listeriolysin, Tween 80 or Tween 20.

45. Use of chitosan in the preparation of an immunogenic composition for inducing or enhancing an immune response, such as a CTL response, against a protein antigen.

46. Use of chitosan and a polypeptide antigen or a variant thereof for the preparation of an immunogenic composition for inducing or enhancing an immune response, such as a CTL response, against the polypeptide antigen.


SEQUENCE LISTING (6 sequences)

SEQ ID NO: 1 - DNA, 2253 bp, Homo sapiens
  CDS (1)..(2253)
  misc_feature (58)..(2253): Human PSM′
atgggtaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaa

SEQ ID NO: 2 - protein, 750 aa, Homo sapiens
MGNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 3 - DNA, 3768 bp, Homo sapiens
  CDS (1)..(3768)
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 4 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 5 - DNA, 648 bp, Homo sapiens
  CDS (1)..(648)
atgggcagcccccgctccgcgctgagctgcctgctgttgcacttgctggttctctgcctccaagcccaggtaactgttcagtcctcacctaattttacacagcatgtgagggagcagagcctggtgacggatcagctcagccgccgcctcatccggacctaccagctctacagccgcaccagcgggaagcacgtgcaggtcctggccaacaagcgcatcaacgccatggcagaagacggagaccccttcgcgaagctcattgtggagaccgatacttttggaagcagagtccgagttcgcggcgcagagacaggtctctacatctgcatgaacaagaaggggaagctaattgccaagagcaacggcaaaggcaaggactgcgtattcacagagatcgtgctggagaacaactacacggcgctgcagaacgccaagtacgagggctggtacatggcctttacccgcaagggccggccccgcaagggctccaagacgcgccagcatcagcgcgaggtgcacttcatgaagcgcctgccgcggggccaccacaccaccgagcagagcctgcgcttcgagttcctcaactacccgcccttcacgcgcagcctgcgcggcagccagaggacttgggccccggagccccgatag

SEQ ID NO: 6 - protein, 215 aa, Homo sapiens
MGSPRSALSCLLLHLLVLCLQAQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR